(19) United States
(12) Patent Application Publication
(10) Pub. No.: US 2022/0256877 A1
(43) Pub. Date: Aug. 18, 2022

(54) CULTURED PLANT-BASED DAIRY ALTERNATIVE

(71) Applicant: Mill It, Inc., Bakersfield, CA (US)

(72) Inventors: Scott William Myers, Bakersfield, CA (US); William Isaac Myers, Orem, UT (US)

(21) Appl. No.: 17/733,681

(22) Filed: Apr. 29, 2022

Related U.S. Application Data
(63) Continuation-in-part of application No. 16/433,272, filed on Jun. 6, 2019.
Application No. 16/433,272 is a continuation-in-part of application No. 16/179,933, filed on Nov. 3, 2018.
Application No. 16/179,933 is a continuation-in-part of application No. 16/179,934, filed on Nov. 3, 2018.
(60) Provisional application No. 62/713,049, filed on Aug. 1, 2018.
(60) Provisional application No. 62/713,049, filed on Aug. 1, 2018.

(51) Int. Cl. A23C 11/10; A23L 33/135; A23L 33/21; A23L 29/00; A23C 9/123; C12N 1/20

(52) U.S. Cl. CPC A23C 11/106; A23L 33/135; C12N 1/205; A23L 29/065; A23C 9/123; A23L 33/21

(57) ABSTRACT

Described herein are composition and method for manufacturing a cultured plant-based dairy alternative based on fermenting ancient grains. In one aspect, the composition contains flour of one or more ancient grains, one or more plant seed butters, one or more plant-based creams, one or more texturizers or stabilizers, and water that is cultured to form a hypoallergenic plant-based dairy alternative containing no added monosaccharides or disaccharides.

### CROSS-REFERENCE TO RELATED APPLICATIONS

This application is a continuation in part of U.S. patent application Ser. No. 16/433,272, filed on Jun. 6, 2019, which is a continuation in part of U.S. patent application Ser. Nos. 16/179,933 and 16/179,934, both filed Nov. 3, 2018, both of which claim priority to U.S. Provisional Patent App. No. 62/713,049, filed on Aug. 1, 2018, each of which is incorporated by reference herein in its entirety.

### TECHNICAL FIELD

Described herein are composition and method for manufacturing a cultured plant-based dairy alternative based on fermenting ancient grains. In one aspect, the composition contains flour of one or more ancient grains, one or more plant seed butters, one or more plant-based creams, one or more texturizers or stabilizers, and water that is cultured to form a hypoallergenic plant-based dairy alternative containing no added monosaccharides or disaccharides.

### BACKGROUND

The food industry has seen the rapid growth in the introduction of cultured and non-cultured plant-based food products typically associated with the dairy industry. Innova Market Insights data shows that the number of new food and beverage launches tracked with plant-based claims in the U.S. witnessed a 54% compound annual growth rate from 2013-2017. Plant-based food product introductions grew at 69% in this five-year period, while plant-based beverages witnessed a 38% growth.

According to the International Food Information Council, 21% of Americans have an improved opinion about the healthfulness of plant-based products, and 69% are trying to consume more plant-based products. While these products roll out across the globe, Americans consumed more plant-based products than any other region in 2017 and this trend is expected to grow.

Many of these early plant-based products were made with soybeans or tree nuts, excluding from their target audience a sizable portion of the US population that suffers from these severe food allergies.

Researchers estimate that up to 15 million Americans have food allergies, including 5.9 million children under age 18. About 30% of these children are allergic to more than one food. Eight major food allergens—milk, eggs, peanuts, tree nuts, wheat (gluten), soy, fin fish, and crustacean shellfish—are responsible for about 90% of the most serious food allergy reactions in the United States.

Among U.S. children, the most common food allergies include peanut, milk, crustacean shellfish, tree nut, egg, fin fish, wheat, and soy. An emerging concern, sesame, is the ninth most common food allergen affecting many children. The Centers for Disease Control and Prevention reports that the prevalence of food allergies in children increased by 50% between 1997-2011. Between 1997-2008, the prevalence of peanut or tree nut allergies more than tripled in U.S. children.

Each year in the U.S., 200,000 people require emergency medical care for allergic reactions to food. Childhood hospitalizations for food allergies tripled between the late 1990s and the mid-2000s. About 40% of children with food allergies have experienced a severe allergic reaction such as anaphylaxis.

In contrast, food intolerance is an abnormal physiological response to a food. For example, lactose intolerance is a digestive condition that occurs when an individual does not produce enough lactase enzymes in the small intestine to digest the lactose in dairy products. Approximately 65% of the human population has a reduced ability to digest lactose after infancy. Lactose intolerance is common in people of East Asian, African, Arab, Jewish, Greek, Italian, and Hispanic descent. The prevalence of lactose intolerance is lowest in populations with histories of dependence on unfermented milk products as an important food source. For example, only about 5% of people of Northern European descent are lactose intolerant.

Symptoms of lactose intolerance include diarrhea, nausea, abdominal cramping, and bloating. Some of the symptoms of lactose intolerance and a food allergy may be similar. However, the distinction between food intolerance food allergies is important. Food intolerance produces unpleasant symptoms, whereas a food allergy reaction can be life threatening.

What is needed is a hypoallergenic cultured plant-based dairy alternative that does not contain any added sugar.

### SUMMARY

One embodiment described herein is a composition comprising: one or more plant-based carbohydrate sources; one or more plant seed butters or creams; one or more plant-based texturizers or stabilizers; a solvent; and one or more microorganism cultures. In one aspect, the one or more plant-based carbohydrate sources comprises millet, sorghum, quinoa, amaranth, einkorn, teff, freekeh, farro (emmer), fonio, spelt, chin seeds, hemp hearts, buckwheat, or combinations thereof. In another aspect, the one or more plant seed butters or creams comprises sunflower seed butter, coconut cream, pumpkin seed butter, watermelon seed butter, hemp seed butter, chia seed butter, flax seed butter, poppy seed butter, or combinations thereof. In another aspect, the one or more plant-based texturizers or stabilizers comprises locust bean gum, gellan gum, xanthan gum, acacia gum, tragacanth gum, guar gum, pectin, agar, or combinations thereof. In another aspect, the solvent comprises water. In another aspect, the one or more microorganism cultures comprises one or more of Bifidobacterium lactis, Lactobacillus acidophilus, Lactobacillus delbrueckii subsp. Bulgaricus, Lactobacillus delbrueckii subsp. Lactis, Lactobacillus helveticus, Lactobacillus kefiranofaciens, Lactobacillus paracasei, Lactobacillus plantarum, Lactococcus lactis, Leuconostoc spp., Pediococcus spp., Streptococcus thermophilus, Kazachstania unispora, Kluyveromyces lactis, Kluyveromyces marxianus, Saccharomyces cerevisiae, Saccharomyces fragilis, Torulaspora delbrueckii, or combinations thereof. In another aspect, the composition further comprises: one or more plant-based proteins comprising pea protein concentrate, potato protein concentrate, bean protein concentrate, sunflower seed protein concentrate, rapeseed protein or combinations thereof; and/or one or more fruit and vegetable soluble fiber concentrates or pomaces or combinations thereof. In another aspect, the composition comprises: 0.1-3.0% by mass of the one or more plant-based carbohydrate sources; 0.2-10.0% by mass of the one or more plant seed butters or creams; 0.1-0.75% by mass of the one or more plant-based texturizers or stabilizers; 80-98% by mass of the solvent; and 0.001-0.005% by mass of the one or more microorganism cultures. In another aspect, the composition comprises: 0.1-3.0% by mass of a flour from millet, sorghum, quinoa, amaranth, einkorn, teff, freekeh, farro (emmer), fonio, spelt, chia seeds, hemp hearts, buckwheat, or combinations thereof; 0.2-10.0% by mass of sunflower seed butter, coconut cream, pumpkin seed butter, watermelon seed butter, hemp seed butter, chia seed butter, flax seed butter, poppy seed butter, or combinations thereof; 0.1-0.75% by mass of one or more of locust bean gum, gellan gum, xanthan gum, acacia gum, tragacanth gum, guar gum, pectin, agar, or combinations thereof; 80-98% by mass of water; and 0.001-0.005% by mass of one or more of Bifidobacterium lactis, Lactobacillus acidophilus, Lactobacillus delbrueckii subsp. Bulgaricus, Lactobacillus delbrueckii subsp. Lactis, Lactobacillus helveticus, Lactobacillus kefiranofaciens, Lactobacillus paracasei, Lactobacillus plantarum, Lactococcus lactis, Leuconostoc spp., Pediococcus spp., Streptococcus thermophilus, Kazachstania unispora, Kluyveromyces lactis, Kluyveromyces marxianus, Saccharomyces cerevisiae, Saccharomyces fragilis, Torulaspora delbrueckii, or combinations thereof. In another aspect, the composition comprises: 0.1-3.0% by mass of a flour from millet, sorghum, or combinations thereof; 0.2-2.0% by mass of sunflower seed butter; 2-8% by mass of coconut cream; 0.1-0.75% by mass of one or more of locust bean gum, gellan gum, xanthan gum, or combinations thereof; 80-98% by mass of water; and 0.001-0.005% by mass of one or more of Bifidobacterium lactis, Lactobacillus acidophilus, Lactobacillus delbrueckii subsp. Bulgaricus, Lactobacillus delbrueckii subsp. Lactis, Lactobacillus plantarum, or Streptococcus thermophilus, or combinations thereof. In another aspect, the composition is vegan, hypoallergenic and contains no added monosaccharides or disaccharides.

Another embodiment described herein is a method for manufacturing a composition described herein, the method comprising: combining and blending the one or more plant-based carbohydrate sources; the one or more plant seed butters or creams; the one or more plant-based texturizers or stabilizers; and the solvent; pasteurizing the blended composition; homogenizing the blended composition; cooling the composition; adding the one or more microorganism cultures to the blended, pasteurized, homogenized, and cooled composition and fermenting the composition until the pH is about 4.4 or lower; and cooling the fermented composition to about 4° C. In one aspect, the combined composition is blended at high shear until the composition is smooth and uniform. In another aspect, the blended composition is pre-heated prior to pasteurization. In another aspect, the blended composition is pasteurized using high temperature, short time (HTST) pasteurization or ultra-high temperature (UHT) pasteurization. In another aspect, the composition is homogenized at a high temperature. In another aspect, the composition is cooled to about ˜40° C. prior to adding the one or more microorganism cultures. In another aspect, the composition is fermented for about 4-8 hours at about ˜40° C. In another aspect, the composition is fermented until the pH is about 4.4 or lower.

Another embodiment described herein is a composition manufactured by any of the methods described herein.

### DESCRIPTION OF THE DRAWINGS

FIG. 1 shows a flow diagram that depicts one exemplary method for making a hypoallergenic, plant-based dairy alternative.

FIG. 2 shows a flow diagram that depicts one alternative exemplary method for creating a hypoallergenic, plant-based dairy alternative that is fermented.

FIG. 3 shows a flow diagram that depicts alternative exemplary methods for creating a hypoallergenic, plant-based dairy alternative based on alternative substrates.

FIG. 4 shows a flow diagram that depicts one alternative method wherein various types of hypoallergenic substrates are combined with water.

FIG. 5-7 show flow diagrams that depict alternative methods for combining water with a hypoallergenic substrate based on a range of ratios of mass percentages.

FIG. 8A-9B show flow diagrams that depict alternative methods for combining water with a plant-based butter and/or cream based on a range of ratios of mass percentages.

FIG. 10 shows a flow diagram that depicts alternative exemplary methods for subjecting the hypoallergenic, dairy alternatives to a preservation process.

FIG. 11-12 show flow diagram that depict alternative methods for inoculating a hypoallergenic, dairy alternative.

FIG. 13 shows a flow diagram that depicts alternative exemplary methods for inoculating a hypoallergenic slurry with a fermentation agent.

FIG. 14 shows a flow diagram that depicts yet additional alternative exemplary methods for inoculating a hypoallergenic slurry with a fermentation agent.

FIG. 15 shows a flow diagram that depicts yet additional alternative exemplary methods for inoculating a hypoallergenic slurry with a fermentation agent.

FIG. 16 shows a flow diagram that depicts yet additional alternative exemplary methods for inoculating a hypoallergenic slurry with a probiotic strain.

FIG. 17A-18D show flow diagrams that depict alternative methods for adjusting the pH of a hypoallergenic slurry.

FIG. 19-20 show flow diagrams that depict alternative exemplary methods wherein a plant-based protein is added to a fermented hypoallergenic slurry.

FIG. 21-22 show flow diagrams that depict alternative exemplary methods wherein a prebiotic soluble fiber is added to a fermented hypoallergenic slurry.

FIG. 23 shows a graph of the pH value versus time for the fermentation of three production runs of the plant-based dairy alternative and the average of the three runs. The cultured plant-based dairy alternative composition as described herein reaches a pH of about 4.4 in about 6 to 6.6 hours after commencing fermentation. Achieving the pH in this time period is surprisingly rapid as compared to typical plant-based fermentation products.

### DETAILED DESCRIPTION

Unless otherwise defined, all technical and scientific terms used herein have the same meaning as commonly understood by one of ordinary skill in the art. For example, any nomenclatures used in connection with, and techniques of, food production and formulation, fermentation, microbiology, biochemistry, and molecular biology, described herein are known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments.

As used herein, the term “hypoallergenic” refers to a product that does not contain allergens, or contains fewer allergens, and does not produce significant allergic reactions in subjects that consume or are contacted by the product.

### Compositions

One embodiment described herein is a composition for a fermented food product. In one aspect, the product is a vegan, non-GMO, hypoallergenic, cultured, plant-based dairy alternative produced from fermenting ancient grains and plant proteins and butters and creams. In one aspect, the composition contains flour of one or more ancient grains, one or more seed butters, one or more plant-based creams, one or more texturizers or stabilizers, and water that is cultured to form a dairy alternative food product. In one aspect, the food product is hypoallergenic and contains no added monosaccharides or disaccharides (sugars). In another aspect, the composition optionally contains plant-based protein concentrates, prebiotic soluble plant fiber, or a combination thereof, Exemplary components of the product are shown in Table 1.

TABLE 1
Exemplary Composition and Mass Percentages

| Component/ | | Mass |
|---|---|---|
| Function | Exemplary Components | Percent (%) |
| Carbohydrate | Ancient grain flours including millet, sorghum, quinoa, | 0.1-3.0 |
| sources | amaranth, teff, freekeh, farro (emmer), fonio, spelt, | |
| | chia seeds, hemp hearts, buckwheat, or combinations | |
| | thereof | |
| Plant seed butters or | Plant-based butters or creams, including sunflower | 0.8-10 |
| creams | seed, pumpkin seed, watermelon seed, hemp seed, | |
| | chia seed, flax seed, poppy seed, coconut cream, or | |
| | combinations thereof | |
| Texturizers or | Gums including locust bean gum, gellan gum, xanthan | 0.1-0.75 |
| stabilizers | gum, acacia gum, tragacanth gum, guar gum, pectin, | |
| | agar, or combinations thereof | |
| Solvent | Water | 80-98 |
| Optional plant-based | Pea protein concentrate, potato protein concentrate, | 0.02-6% |
| protein concentrates | bean protein concentrate, sunflower seed protein | (0.01-3% |
| and/or prebiotic | concentrate, rapeseed protein or combinations thereof | each) |
| soluble plant fibers | Fruit or vegetable soluble fiber concentrates or | |
| | pomaces or combinations thereof | |
| Microorganism | Bifidobacterium lactis, Lactobacillus acidophilus, | 0.001- |
| cultures | Lactobacillus delbrueckii subsp. Bulgaricus, | 0.005% |
| (fermentation agents) | Lactobacillus delbrueckii subsp. Lactis, Lactobacillus | |
| | helveticus, Lactobacillus kefiranofaciens, Lactobacillus | |
| | paracasei, Lactobacillus plantarum, Lactococcus lactis, | |
| | Leuconostocspp., Pediococcus spp., Streptococcus | |
| | thermophilus, Kazachstania unispora, Kluyveromyces | |
| | lactis, Kluyveromyces marxianus, Saccharomyces | |
| | cerevisiae, Saccharomyces fragilis, Torulaspora | |
| | delbrueckii, or combinations thereof. | |

In one embodiment the composition comprises one or more carbohydrate sources. In one aspect, the carbohydrate source is an extract or flour from one or more ancient grains. Ancient grains include grains and pseudocereals that have not undergone selective breeding. Exemplary ancient grains include millet, sorghum, quinoa, amaranth, teff, freekeh, farro (emmer), fonio, spelt, chia seeds, hemp hearts, buckwheat, or combinations thereof. These ancient grains are hypoallergenic and provide complex carbohydrates which can be utilized by the cultures during fermentation. In one embodiment, the ancient grains comprise flours of millet, sorghum, or a combination thereof. In the composition, the ancient grains are typically present in an amount of about 0.1% to about 5% by mass, including all integers within the range. In one aspect, the ancient grains or components thereof comprise 0.1% to about 3% by mass of the composition, including all integers within the range. One or more of the ancient grains or components thereof can comprise about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0% by mass of the composition. In one aspect, one or more of the ancient grains or components thereof can comprise about 0.1-1.0%; 0.2-0.8%, 0.3-0.6%, 0.4-0.5%, 0.1-1%, 0.1-2%, 0.1-3%, 0.1-4%, 0.2-1%, 0.3-1%, 0.4-1%, 0.5-1%, 0.6-1%, 0.7-1%, 0.2-0.6, 0.2-0.8, 0.3-0.3-0.6%, 0.3-0.8%, 0.4-0.5%, 0.4-0.6%, 0.4-0.8%, 0.5-0.6%, 0.5-0.7%, 0.5-0.8%, or about 0.6-0.8% by mass. In one aspect, the one or more of the ancient grains or components thereof comprises about 0.2-0.8% by mass of the composition.

The composition also contains plant seed butters and creams. In one aspect, the plant seed butters and creams comprise high amounts of polyunsaturated fatty acids and monounsaturated fatty acids in addition to carbohydrates, proteins, fiber. In another aspect, the plant seed butters and creams are hypoallergenic. In one embodiment, the plant seed butters and creams comprise sunflower seed butter, pumpkin seed butter, watermelon seed butter, hemp seed butter, chia seed butter, flax seed butter, poppy seed butter, coconut cream, or combinations thereof. In one embodiment, the plant seed butter or cream comprises sunflower butter. In one embodiment, the plant seed butter or cream comprises coconut cream. Coconut cream is desirable because it has dairy notes and provides the composition a dairy flavor profile. In one embodiment, the plant seed butter or cream comprises a combination of sunflower butter and coconut cream. The plant seed butter or cream comprises about 0.8% to about 10% by mass of the composition, including all integers within the range. In one aspect, the plant seed butter or cream can comprise about 0.8, 0.9, 1.0, 1.1, 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, or about 10.0% by mass of the composition. In another aspect, the plant seed butter or cream comprises about 0.8-10%, 0.9-8%, 1.0-7%, 1.0-6%, 1.0-1.4%, 1.0-1.5%,1.0-1.6%, 1.2-1.5%, 1.1-1.5%, 1.1-1.4%, 1.2-1.4%, 0.9-2% 1.0-2%, 1.1-2%, 1.2-2%, 1.3-2%, 1.4-2%, 1.5-2%, 1.6-2%, 2-3, 2-4, 2-5, 2-6, 2-7, 2.5-5, 2.5-7, 2.5-8, 3-5, 3-7, 3-8, 4-5, 4-6, 4-8, 5-7, 5-8, 6-7, or about 6-8% by mass. In one embodiment, the one or more plant seed butters comprise about 1% to about 1.5% by mass of the composition and the one or more plant creams comprise about 3 to about 5% by mass of the composition, for a total percentage of about 4-7% by mass, including all integers within the range.

The composition also contains one or more plant-based texturizers or stabilizers. In one embodiment the plant-based texturizers or stabilizers comprise one or more of locust bean gum, gellan gum, xanthan gum, acacia gum, tragacanth gum, guar gum, pectin, agar, or combinations thereof. In one embodiment the texturizers or stabilizers comprise one or more of locust bean gum, gellan gum, xanthan gum. The plant-based texturizers or stabilizers comprise about 0.1% to about 1% by mass of the composition, including all integers within the range. In one aspect, the plant-based texturizers or stabilizers comprise about 0.1, 0.2, 0.3, 0.4, 0.5, 0.6, 0.7, 0.8, 0.9, to about 1.0 by mass of the composition. In another aspect, the plant-based texturizers or stabilizers comprise about 0.1-0.8, 0.2-0.7, 0.3-0.5, 0.1-0.2, 0.1-0.3, 0.1-0.5, 0.1-0.7, 0.1-0.8, 0.2-0.3, 0.2-0.4, 0.2-0.5, 0.2-0.6, 0.2-0.8, 0.3-0.6, 0.4-0.8, 0.5-1, 0.6-1, or about 0.8-1% by mass of the composition. In one aspect, the plant-based texturizers or stabilizers comprise about 0.2 to about 0.5% by mass of the composition.

The composition contains a solvent. In one embodiment the solvent is purified water. The composition comprises water in an amount of about 80% to about 98% by mass of the composition, including all integers within the range. In one aspect, the water comprises about 80, 81, 82, 83, 84, 85, 86, 87, 88, 89, 90, 91, 92, 93, 94, 95, 96, 97, or about 98% by mass of the composition. In one aspect, the water comprises about 92% to about 95% of the composition by mass.

The composition comprises one or more microorganism cultures or fermentation agents. The culture can comprise bacteria, yeasts, or combinations thereof. Exemplary microorganisms for fermentation comprise one or more of Bifidobacterium lactis, Lactobacillus acidophilus, Lactobacillus delbrueckii subsp. Bulgaricus, Lactobacillus delbrueckii subsp. Lactis, Lactobacillus helveticus, Lactobacillus kefiranofaciens, Lactobacillus paracasei, Lactobacillus plantarum, Lactococcus lactis, Leuconostoc spp., Pediococcus spp., Streptococcus thermophilus, Kazachstania unispora, Kluyveromyces lactis, Kluyveromyces marxianus, Saccharomyces cerevisiae, Saccharomyces fragilis, Torulaspora delbrueckii, or combinations thereof. In one aspect, the microorganism cultures can comprise about 0.001% to about 0.01% by mass of the composition, including all integers within the range. In another aspect, the culture can comprise about 0.001, 0.002, 0.003, 0.004, 0.005, 0.006, 0.007, 0.008, 0.009, or about 0.01% by mass of the composition. In another aspect the culture comprises 0.001-0.002, 0.001-0.003, 0.001-0.004, 0.001-0.005, 0.001-0.006, 0.001-0.007, 0.001-0.008, 0.001-0.009, 0.002-0.003, 0.002-0.005, 0.002-0.008, 0.003-0.005, 0.001-0.008, 0.004-0.006, 0.004-0.008, or 0.005-0.008% by mass of the composition. In one aspect, the culture comprises about 0.002% to about 0.004% by mass of the composition.

The composition can optionally contain plant-based protein concentrates or prebiotic soluble fiber sources. Exemplary plant-based protein concentrations comprise pea protein concentrate, potato protein concentrate, bean protein concentrate, sunflower seed protein concentrate, rapeseed protein or combinations thereof. Exemplary plant-based fiber sources include fruit and vegetable fiber concentrates or pomaces or combinations thereof. The composition can comprise combined plant-based protein concentrates and/or fiber sources at about 0.02 to about 6% by mass of the composition, including all integers within the range (e.g., about 0.01 to about 3% of one or more protein concentrates and about 0.25 to about 3% of one or more prebiotic soluble fiber sources). In one aspect, the composition comprises combined plant-based protein concentrates and/or prebiotic soluble fiber sources at about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.4, 5.6, 5.8, or about 6.0% by mass of the composition. In another aspect, the composition comprises combined plant-based protein concentrates and/or prebiotic soluble fiber sources at about 0.001-5, 0.05-5, 0.1-5, 0.01-1, 0.05-1, 0.5-5, 0.5-4, 0.5-3, 0.5-2, 0.5-1, 1-2, 1-3, 1-4, 1-5, 2-3, 2-4, 2-5, 3-4, 3-5, or about 4-5% by mass of the composition. In one aspect, the composition comprises combined plant-based protein concentrates and/or prebiotic soluble fiber sources at about 0.5% to about 3% by mass of the composition.

In one embodiment the plant-based dairy alternative comprises about 0.2-0.8% by mass of one or more of the ancient grains or components thereof; about 4-8% by mass of one or more plant seed butters or creams; about 0.2-0.5% by mass of one or more plant-based texturizers or stabilizers; about 92-95% by mass water; and about 0.002-0.004% by mass of one or more microorganism cultures. In one aspect, the ancient grains comprise millet and sorghum flours; the plant seed butter or cream comprises sunflower seed butter and coconut cream; the texturizers or stabilizers comprise one or more of locust bean gum, gellan gum, xanthan gum; and the microorganism comprises one or more of Bifidobacterium lactis, Lactobacillus acidophilus, Lactobacillus delbrueckii subsp. Bulgaricus, Lactobacillus delbrueckii subsp. Lactis, Lactobacillus helveticus, Lactobacillus kefiranofaciens, Lactobacillus paracasei, Lactobacillus plantarum, Lactococcus lactis, Leuconostoc spp., Pediococcus spp., or Streptococcus thermophilus. Optionally, the plant-based dairy alternative can comprise about 0.5-3.0% of each of plant-based protein concentrates and/or prebiotic soluble plant-based fiber sources.

### Methods

Another embodiment described herein is a method for manufacturing a cultured plant-based dairy alternative. The manufacturing method comprises combining components into a high-shear mixer, blending the batch at high shear until it is smooth, and the texturizers or stabilizers are dispersed, and no clumps are formed. The composition is transferred to a batch kettle and mixed with gentle agitation until the pasteurization/homogenization steps. Prior to pasteurization, the composition is preheated to 40-48° C. (107-117° F.). Pasteurization can be performed using either high temperature short time pasteurization (HTST) or ultra-high temperature pasteurization (UHT). For HTST, the composition is pasteurized at 93° C. (200° F.) followed by homogenization at 93° C. (200° F.), 500/1500 psi for 7 minutes. For UHT, the composition is pasteurized 138-150° C. (280-302° F.) for one or two seconds, followed by homogenization at 93° C. (200° F.), 500/1500 psi for up to 3 seconds. Following pasteurization/homogenization, the mixture is cooled to about 40° C. (106° F.). The microorganism cultures are added and mixed and the composition is allowed to ferment at ˜40° C. (106° F.) for about 4-8 hours or until the pH reaches 4.4 or lower. Following culturing, the composition is transferred to a cooling tank and held at 4° C. (39° F.).

As used herein the term “hypoallergenic substrate” refers to one or more carbohydrate sources, typically one or more ancient grains or flours or extracts thereof, that are hypoallergenic.

As used herein the term “hypoallergenic-slurry” refers to a composition comprising one or more “hypoallergenic substrates” or carbohydrate sources, one or more plant seed butters or creams, one or more plant-based texturizers or stabilizers, or combinations thereof, and one or more solvents.

As used herein the term “fermented hypoallergenic-slurry” refers to a composition comprising one or more “hypoallergenic substrates” or carbohydrate sources, one or more plant seed butters or creams, one or more texturizers or stabilizers, or combinations thereof, and one or more solvents that has optionally been pasteurized and homogenized, cooled, and then inoculated with one or more microorganism cultures and allowed to ferment for a period of time. In one aspect, the period of time for fermentation is from about 4 to about 8 hours.

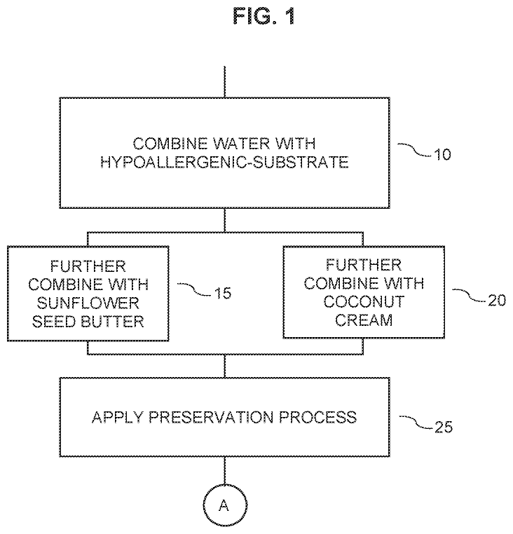

FIG. 1 shows a flow diagram that depicts one exemplary method for making a hypoallergenic, plant-based dairy alternative. In one embodiment the hypoallergenic, plant-based dairy alternative is a cultured buttermilk product. According to this exemplary method, a hypoallergenic, plant-based dairy alternative is made by creating a hypoallergenic-slurry by combining water with a hypoallergenic substrate (Step 10). In continuation of creating the hypoallergenic-slurry, the water is further combined with a sunflower seed butter (Step 15). In one alternative method, the hypoallergenic-slurry is created by further combining the water with a coconut cream (Step 20). In this exemplary method, the hypoallergenic-slurry is then subject to a preservation process (Step 25). It should likewise be appreciated that, according to various alternative exemplary methods, a stabilizer is also added to the hypoallergenic-slurry in order to help retard separation of a plant-based butter or cream from the water or the hypoallergenic substrate. It should be appreciated that the hypoallergenic plant-based cultured dairy alternative created by the method described herein is useful as a base for many varying products, including, for example, buttermilk products, yogurt, kefir, or salad dressings.

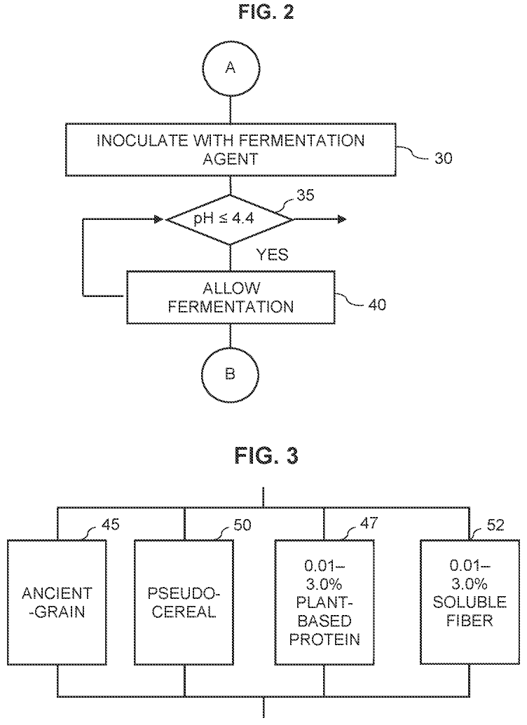

FIG. 2 shows a flow diagram that depicts one alternative exemplary method for creating a hypoallergenic, plant-based dairy alternative that is also fermented. The hypoallergenic, plant-based dairy alternative heretofore described is also useful as a basis for other products, including cultured substitutes for dairy products. According to one alternative exemplary method, making a hypoallergenic, plant-based dairy alternative further comprises an included step for inoculating the hypoallergenic-slurry with a fermentation agent (Step 30). Once the hypoallergenic-slurry is inoculated with the fermentation agent, it is allowed to ferment (Step 40) until it reaches a pH of 4.4 or lower (Step 35).

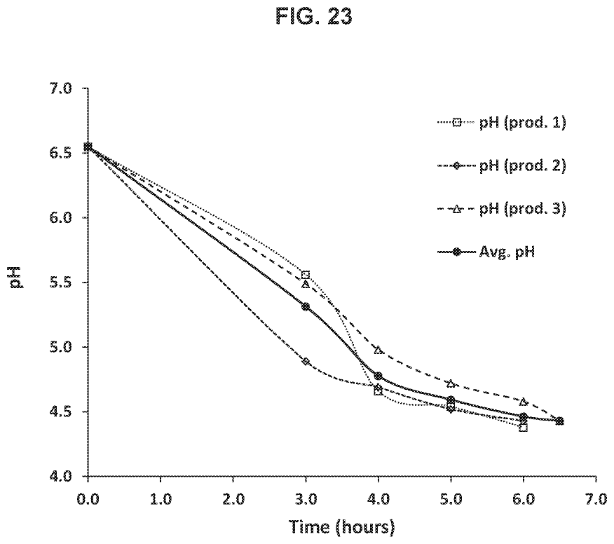

Surprisingly, there exists a synergy in the stated formulation of the hypoallergenic-slurry. The unique combination of the plant-based butter, plant-based cream, and the ancient grains or pseudocereals surprising facilitates fermentation at a faster rate than expected and achieves a lower pH than expected or when the individual components are fermented separately. See FIG. 23, which shows that exemplary compositions as described herein reach a pH of about 4.4 in about 6 hours on average. This is much faster than typical plant-based compositions inoculated with the standard fermentation inoculation culture dose. Many plant-based compositions also require higher doses of fermenting organisms to achieve the desired pH in reasonable time.

FIG. 3 shows a flow diagram that depicts alternative exemplary methods for creating a hypoallergenic, plant-based dairy alternative based on alternative substrates. The term hypoallergenic “plant milk” or “non-dairy milk” refers to a liquid that resembles dairy milk but is made from hypoallergenic plant-based ingredients. Said hypoallergenic plant-based substrate comprises at least one or more of proteins, fats, carbohydrates, vitamins, or minerals. It should be appreciated that dairy allergens, lactose, cholesterol, soy, or tree nut allergens are not included. According to one alternative exemplary embodiment, the method for creating a hypoallergenic, plant-based dairy alternative comprises a step wherein combining the water with a hypoallergenic substrate comprises combining the water with one or more ancient grains (Step 45). In yet another alternative exemplary embodiment, the method for creating a hypoallergenic, plant-based dairy alternative comprises a step wherein combining the water with a hypoallergenic substrate comprises combining the water with a pseudocereal (Step 50). In another alternative exemplary embodiment, the method for creating a hypoallergenic, plant-based dairy alternative comprises a step of combining water with one or more ancient grains (Step 45) and/or one or more pseudocereals (Step 50), and optionally one or more plant based proteins (Step 47) and/or one or more plant-based soluble fibers (Step 52).

In these exemplary embodiments, the fermented, hypoallergenic, plant-based dairy alternative provides a substrate suitable for use as a base in various food products, including salad dressings. The fermented, hypoallergenic, plant-based dairy alternative can also be used to create non-dairy alternatives for products such as buttermilk or yogurt. Salad dressings, yogurt, and buttermilk are only illustrative examples and other products can be produced from the fermented hypoallergenic, plant-based dairy alternative.

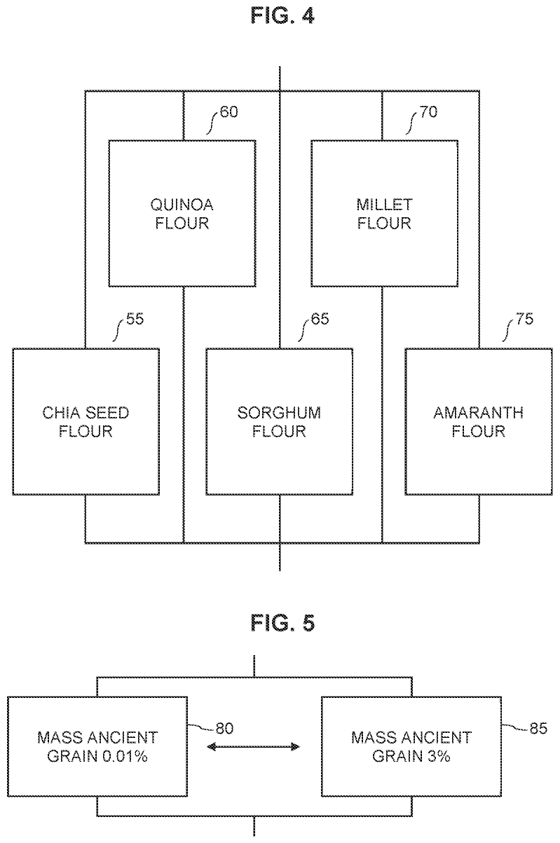

FIG. 4 shows a flow diagram that depicts one alternative method wherein various types of hypoallergenic substrates are combined with water. According to one alternative exemplary method, a step is included for combining a chia seed flour (Step 55) with water. In yet another alternative exemplary embodiment, an included step provides for combining quinoa flour (Step 60) with water. In yet another alternative method, sorghum flour is combined with water (Step 65) in an included step. Millet flour (Step 70) is used in yet an alternative included step for combining water with a hypoallergenic substrate. Amaranth flour (Step 75) is used in yet another alternative included step for combining water with a hypoallergenic substrate. Other potential ancient grains or pseudocereals or flours or extracts thereof include teff, freekeh, farro (emmer), fonio, spelt, chia seeds, hemp hearts, or buckwheat. It should be appreciated that these various types of alternative ingredients, used in corresponding method steps, provide a base that is suitable for fermentation or as a base for other non-dairy, dairy alternatives.

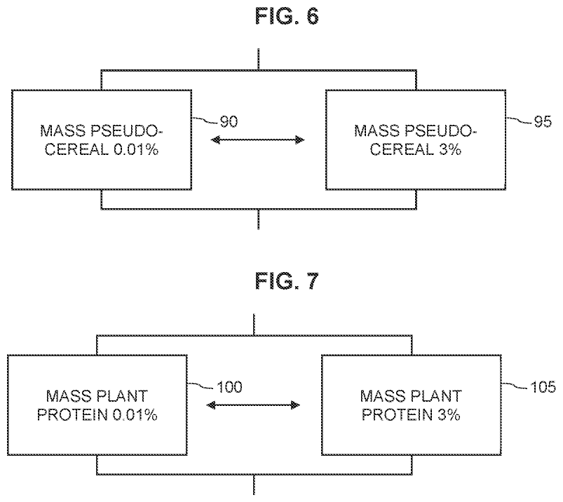

FIG. 5-7 show flow diagrams that depict alternative methods for combining water with a hypoallergenic substrate based on a range of ratios of mass percentages. hi one alternative exemplary embodiment, combining water with a hypoallergenic substrate comprises combining with the water an ancient grain in a ratio by mass percentage wherein the mass percentage of the ancient grain is at a minimum of 0.01% of the water (Step 80). Various ratios beginning at 0.01% and running through 3.0% of the mass percentage of water in the mass percentage of an ancient grain (Step 85) is to be included in the methods described herein. In one aspect the water is about 80-98% by mass and the ancient grain is about 0.1-3.0% by mass.

In yet another alternative exemplary embodiment, combining water with a hypoallergenic substrate comprises combining with the water a pseudocereal in a ratio by mass percentage wherein the mass percentage of the pseudocereal is at a minimum of 0.01% of the water (Step 90). Various ratios beginning at 0.01% and running through 3.0% of the mass of water in the mass percentage of a pseudo-cereal (Step 95) is to be included in the methods described herein. In one aspect the water is about 80-98% by mass and the pseudocereal is about 0.1-3.0% by mass.

In another embodiment, the composition comprises a plant-based protein. Accordingly, one alternative method comprises combining water with a plant-based protein in a ratio by mass percentage wherein the mass percentage of the plant-based protein is at a minimum of 0.01% of the water (Step 100). Various ratios beginning at 0.01% and running through 3.0% of the mass percentage of water in the mass percentage of a plant-based protein (Step 105) can be included in the methods described herein. In one aspect the water is about 80-98% by mass and the plant-based protein is about 0.1-3.0% by mass.

In another embodiment, the composition comprises a soluble fiber. In one alternative method comprises adding at least 0.01% (by mass) of soluble fiber to the composition. In one aspect, the method comprises adding up to 3.0% (by mass) of soluble fiber to the composition. In one aspect the water is about 80-98% by mass and the soluble fiber is about 0.1-3.0% by mass.

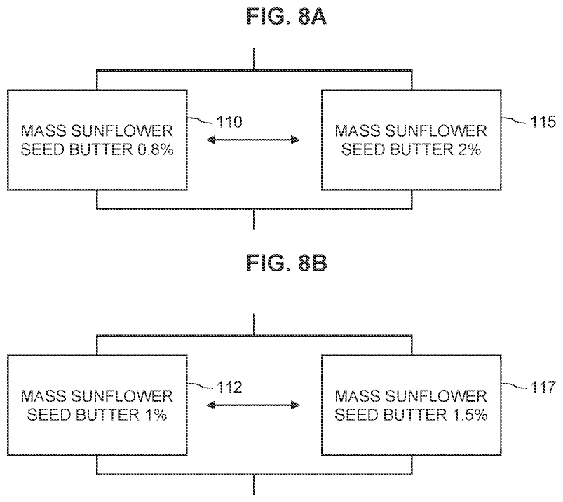

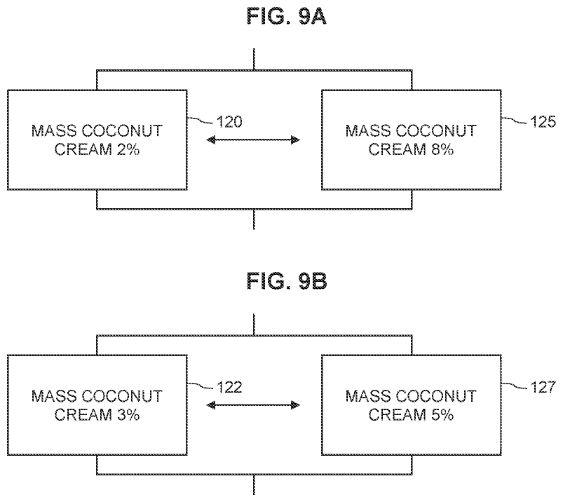

FIG. 8A-9B show flow diagrams that depict alternative methods for combining water with a plant-based butter and/or a cream based on a range of ratios of mass percentages. In one aspect of Step 100, water is combined with a sunflower seed butter at a mass percentage ranging from 0.8% (Step 110) to 2% (Step 115). One alternative method provides for combining water with sunflower seed butter in a ratio by mass percentage wherein the mass percentage of the sunflower seed butter is at a minimum of 0.01% of the water. Various ratios beginning at 0.01% and running through 5.0% of the mass percentage of water in the mass percentage of a sunflower seed butter are to be included in the methods described herein. In one aspect of Step 105, water is combined with a sunflower seed butter at a mass percentage ranging from 1% (Step 112) to 1.5% (Step 117). According to yet another variation of the present method, various ratios beginning at 4.9% and running through 10.0% of the mass percentage of water in the mass percentage of a sunflower seed butter are to be included in the methods described herein. It should be appreciated that, although a preferred method provides for adding an amount of sunflower seed butter up to 5% of the mass percentage of the water, other variations of the present method are acceptable where the amount of sunflower seed butter combined with the water in an amount of up to 10% of the mass percentage of the water. It should be appreciated that, in these variations of the method, much more stabilizer is required, relative to the preferred methods, to help retard the separation of the sunflower seed butter from the water. In one aspect, the water is about 80-98% by mass and the sunflower seed butter is about 0.8-2.0% by mass.

In another aspect of Step 100, water is combined with a coconut cream at a mass percentage ranging from 2% (Step 120) to 8% (Step 125). One alternative method provides for combining water with coconut cream in a ratio by mass percentage wherein the mass percentage of the coconut cream is at a minimum of 0.01% of the water. Various ratios beginning at 0.01% and running through 5.0% of the mass percentage of water in the mass percentage of a coconut cream are to be included in the methods described herein. In another aspect of Step 105, water is combined with a coconut cream at a mass percentage ranging from 3% (Step 122) to 5% (Step 127). According to yet another variation of the present method, various ratios beginning at 4.9% and running through 10.0% of the mass percentage of water in the mass percentage of a coconut cream are to be included in the methods described herein. It should be appreciated that, although a preferred method provides for adding an amount of coconut cream up to 5% of the mass percentage of the water, other variations of the present method are acceptable where the amount of coconut cream combined with the water in an amount of up to 10% of the mass percentage of the water. It should be appreciated that, in these variations of the method, much more stabilizer is required, relative to the preferred methods, to help retard the separation of the coconut cream from the water. In one aspect, the water is about 80-98% by mass and the sunflower seed butter is about 2-8.0% by mass.

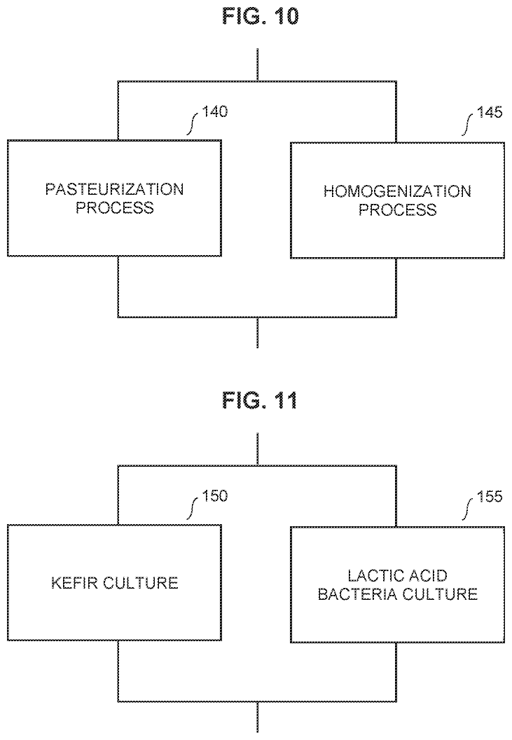

FIG. 10 shows a flow diagram that depicts alternative exemplary methods for subjecting the hypo-allergenic, dairy alternatives to a preservation process. According to one alternative exemplary method, preservation is accomplished by a method step for subjecting the products to a pasteurization process (Step 140). In yet another alternative exemplary method, preservation is accomplished by a method step for subjecting the product to a homogenization process (Step 145).

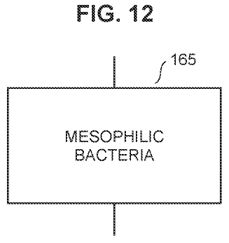

FIG. 11-12 show flow diagrams that depict alternative methods for inoculating a hypoallergenic, dairy alternative. It has been discovered that a hypoallergenic, plant-based substrate can be fermented using lactic acid and other mesophilic bacteria used for making yogurt, buttermilk, or kefir. It has also been discovered that this plant-based substrate can be fermented without the specific addition of a monosaccharide or disaccharide. Accordingly, one alternative exemplary method provides for inoculating the hypoallergenic-slurry with the kefir culture (Step 150). And in yet another alternative exemplary method step, the hypoallergenic slurry is inoculated with a lactic acid bacteria culture (Step 155). And in yet another alternative exemplary method, the hypoallergenic slurry is inoculated with a mesophilic bacteria culture (Step 165).

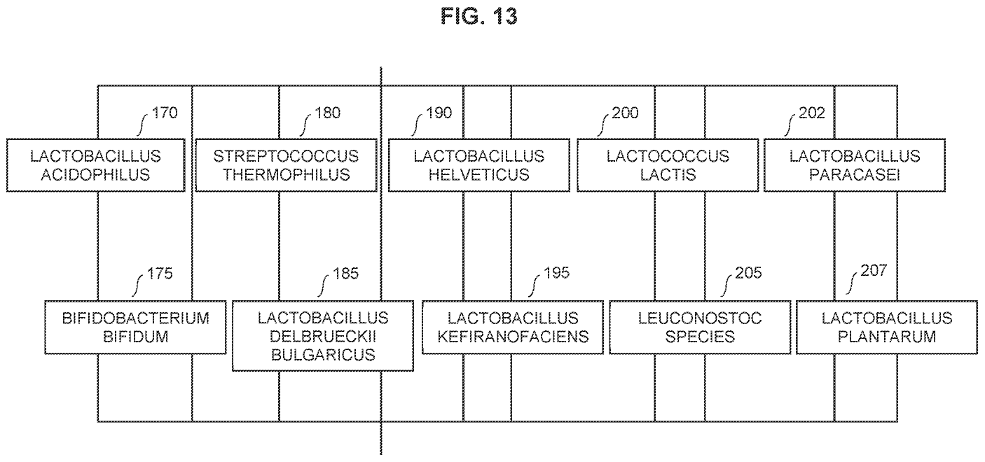

FIG. 13 shows a flow diagram that depicts alternative exemplary methods for inoculating a hypoallergenic slurry with a fermentation agent. According to these various alternative exemplary methods, the included step of inoculating a hypoallergenic slurry comprises inoculating a hypoallergenic slurry with a fermentation agent including at least one or more of Lactobacillus acidophilus (Step 170), Bifidobacterium bifidum (Step 175), Streptococcus thermophilus (Step 180), Lactobacillus delbrueckii bulgaricus (Step 185), Lactobacillus helveticus (Step 190), Lactobacillus kefiranofaciens (Step 195), Lactococcus lactis (Step 200), Leuconostoc species (Step 205), Lactobacillus paracasei (Step 202), Lactobacillus plantarum (Step 207), or combinations thereof. In one aspect the fermentation agent is about 0.001-0.005% by mass of the composition.

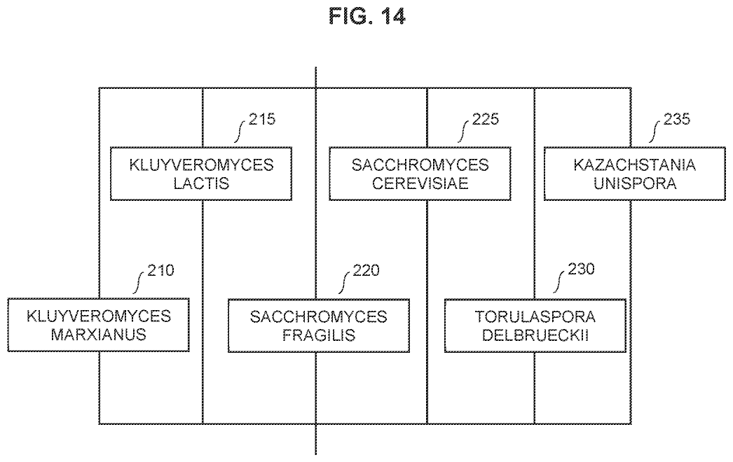

FIG. 14 shows a flow diagram that depicts yet additional alternative exemplary methods for inoculating a hypoallergenic slurry with a fermentation agent. According to these various alternative exemplary methods, the included step of inoculating a hypoallergenic slurry comprises inoculating a hypoallergenic slurry with a fermentation agent including at least one or more of Kluyveromyces marxianus (Step 210), Kluyveromyces lactis (Step 215), Saccharomyces fragilis (Step 220), Saccharomyces cerevisiae (Step 225), Torulaspora delbrueckii (Step 230), Kazachstania unispora (Step 235), or combinations thereof. In one aspect the fermentation agent is about 0.001-0.005% by mass of the composition.

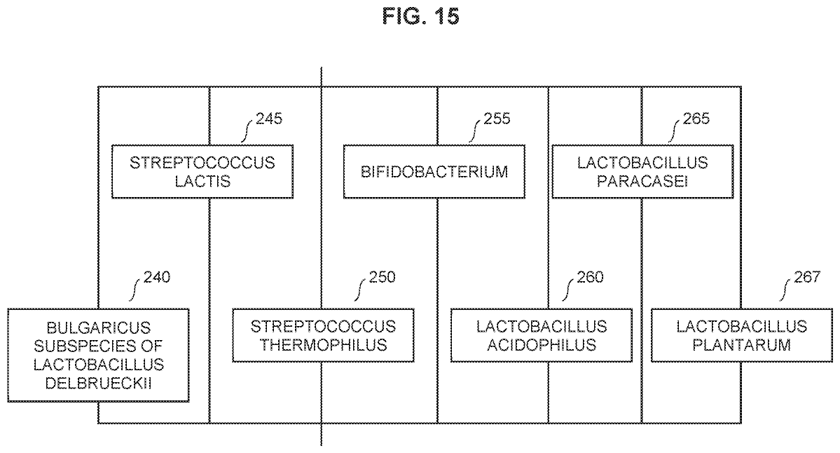

FIG. 15 shows a flow diagram that depicts yet additional alternative exemplary methods for inoculating a hypoallergenic slurry with a fermentation agent. According to these various alternative exemplary methods, the included step of inoculating a hypoallergenic slurry comprises inoculating a hypoallergenic slurry with a fermentation agent including at least one or more of Lactobacillus delbrueckii bulgaricus (Step 240), Streptococcus lactis (Step 245), Streptococcus thermophilus (Step 250), Bifidobacterium (Step 255), Lactobacillus acidophilus (Step 260), Lactobacillus paracasei (Step 265), Lactobacillus plantarum (Step 267), or combinations thereof. In one aspect the fermentation agent is about 0.001-0.005% by mass of the composition.

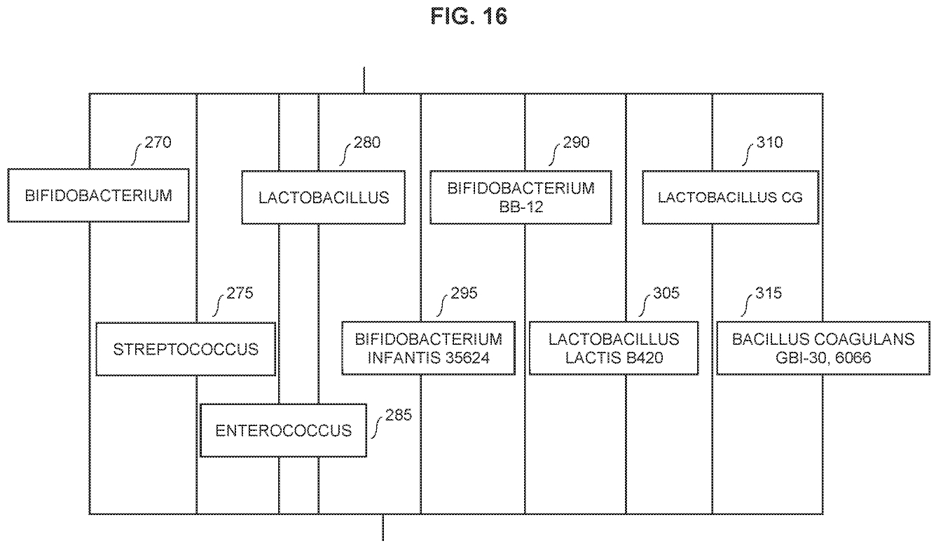

FIG. 16 shows a flow diagram that depicts yet additional alternative exemplary methods for inoculating a hypoallergenic slurry with a probiotic strain. According to these various alternative exemplary methods, the included step of inoculating a hypoallergenic slurry comprises inoculating a hypoallergenic slurry with a probiotic strain including at least one or more of Bifidobacterium (Step 270), Streptococcus (Step 275), Lactobacillus (Step 280), Enterococcus (Step 285), Bifidobacterium BB-12 (Step 290), Bifidobacterium infantis 35624 (Step 295), Lactobacillus lactis B420 (Step 305), Lactobacillus GG (Step 310), Bacillus coagulans GBI-30, 6086 (Step 315), or combinations thereof. In one aspect the probiotic strain is about 0.001-0.005% by mass of the composition.

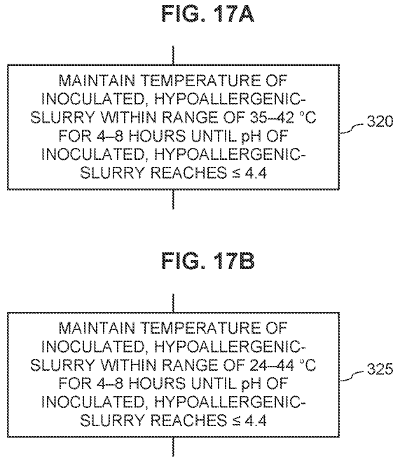

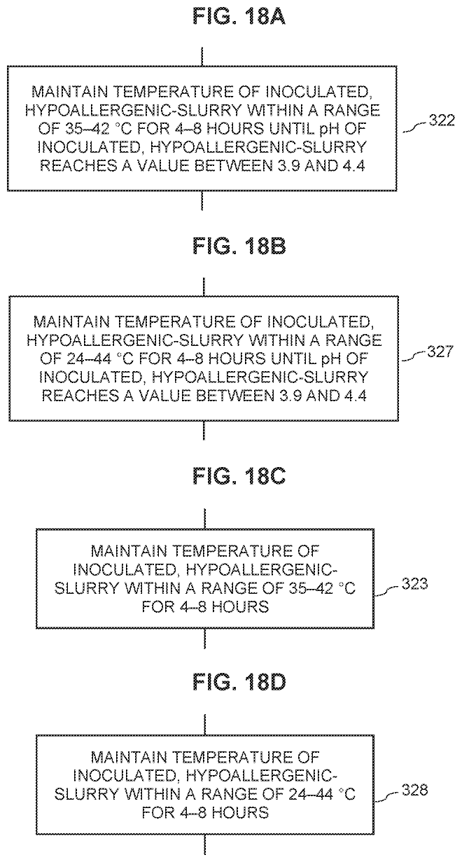

FIG. 17A-18D show flow diagrams that depict alternative methods for adjusting the pH of a hypoallergenic slurry. According to one alternative exemplary method, a step is included for maintaining the temperature of the hypoallergenic slurry within a range substantially between 35-41° C. (95-106° F.) until the pH of the hypoallergenic slurry reaches about 4.4 or lower (Step 320). According yet another alternative exemplary method, a step is included for maintaining the temperature of the hypoallergenic slurry within a range substantially between 23-43° C. (75-110° F.) until the pH of the hypoallergenic slurry reaches about 4.4 or lower (Step 325). In one aspect, the hypoallergenic slurry reaches a pH of about 4.4 or lower in about 6 to about 6.5 hours.

According to other various exemplary methods, a step is included for maintaining the temperature of the hypoallergenic slurry within a range substantially between 35-41° C. (95-106° F.) until the pH of the hypoallergenic slurry reaches a value of about 3.8 to about 4.6, including all integers and end points of this range (Step 322). According yet another alternative exemplary method, a step is included for maintaining the temperature of the hypoallergenic slurry within a range substantially between 23-43° C. (75-110° F.) until the pH of the hypoallergenic slurry reaches a value of 3.9 through 4.6, inclusive of the end points of this range (Step 327). In one aspect, the hypoallergenic slurry reaches a pH of about 3.9-4.4 in about 4 to about 7 hours.

According to other various exemplary methods, a step is included for maintaining the temperature of the hypoallergenic slurry within a range substantially between 35-41° C. (95-106° F.) for an amount of time between 3.9 and 8.1 hours (Step 323). In another alternative method, a step is included for maintaining the temperature of the hypoallergenic slurry within a range substantially between 23-43° C. (75-110° F.) for an amount of time between 3.9 and 8.1 hours (Step 328). In one aspect, the hypoallergenic slurry is maintained at a temperature range substantially between 23-43° C. (75-110° F.) for about 4 to about 6.5 hours.

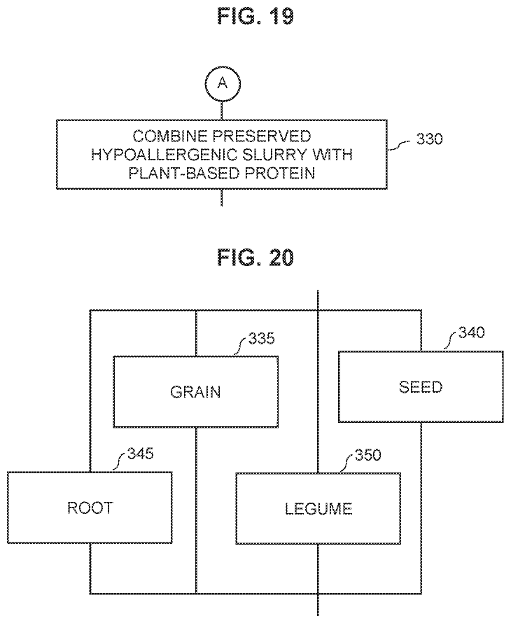

FIG. 19-20 show flow diagrams that depict alternative exemplary methods wherein a plant-based protein is added to a fermented hypoallergenic slurry. It should be appreciated that, according to this alternative exemplary method, an additional included step provides for combining a plant-based protein with a fermented hypoallergenic slurry (Step 330). According to various alternative exemplary methods, the included step for combining a plant-based protein with the fermented hypoallergenic slurry comprises combining at least one or more of a grain (Step 335), a seed (Step 340), a root (Step 345), a legume (Step 350) or combinations thereof as protein sources. Exemplary protein sources include pea protein concentrate, potato protein concentrate, bean protein concentrate, sunflower seed protein concentrate, rapeseed protein or combinations thereof. In addition to or alternatively, fruit and vegetable fiber concentrates or pomaces or combinations thereof can also be added to the fermented hypoallergenic slurry.

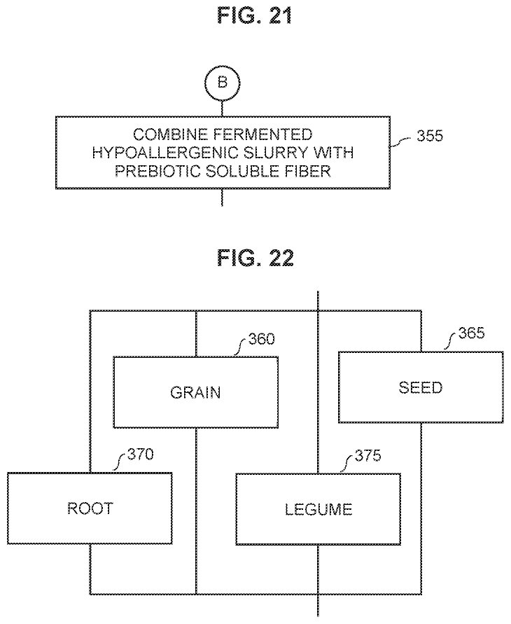

FIG. 21-22 show flow diagrams that depict alternative exemplary methods wherein a prebiotic fiber is added to a fermented hypoallergenic slurry. It should be appreciated that, according to this alternative exemplary method, an additional included step provides for combining a prebiotic soluble fiber with a fermented hypoallergenic slurry (Step 355). According to various alternative exemplary methods, the included step for combining a prebiotic soluble fiber with the fermented hypoallergenic slurry comprises combining at least one or more of a grain (Step 360), a seed (Step 365) a root (Step 370), a legume (Step 375) or combinations thereof as prebiotic soluble fiber sources.

One embodiment described herein is a composition comprising: one or more plant-based carbohydrate sources; one or more plant seed butters or creams; one or more plant-based texturizers or stabilizers; a solvent; and one or more microorganism cultures. In one aspect, the one or more plant-based carbohydrate sources comprises millet, sorghum, quinoa, amaranth, einkorn, teff, freekeh, farro (emmer), fonio, spelt, chia seeds, hemp hearts, buckwheat, or combinations thereof. In another aspect, the one or more plant seed butters or creams comprises sunflower seed butter, coconut cream, pumpkin seed butter, watermelon seed butter, hemp seed butter, chia seed butter, flax seed butter, poppy seed butter, or combinations thereof. In another aspect, the one or more plant-based texturizers or stabilizers comprises locust bean gum, gellan gum, xanthan gum, acacia gum, tragacanth gum, guar gum, pectin, agar, or combinations thereof. In another aspect, the solvent comprises water. In another aspect, the one or more microorganism cultures comprises one or more of Bifidobacterium lactis, Lactobacillus acidophilus, Lactobacillus delbrueckii subsp. Bulgaricus, Lactobacillus delbrueckii subsp. Lactis, Lactobacillus helveticus, Lactobacillus kefiranofaciens, Lactobacillus paracasei, Lactobacillus plantarum, Lactococcus lactis, Leuconostoc spp., Pediococcus spp., Streptococcus thermophilus, Kazachstania unispora, Kluyveromyces lactis, Kluyveromyces marxianus, Saccharomyces cerevisiae, Saccharomyces fragilis, Torulaspora delbrueckii, or combinations thereof. In another aspect, the composition further comprises: one or more plant-based proteins comprising pea protein concentrate, potato protein concentrate, bean protein concentrate, sunflower seed protein concentrate, rapeseed protein or combinations thereof; and/or one or more fruit and vegetable soluble fiber concentrates or pomaces or combinations thereof. In another aspect, the composition comprises: 0.1-3.0% by mass of the one or more plant-based carbohydrate sources; 0.2-10.0% by mass of the one or more plant seed butters or creams; 0.1-0.75% by mass of the one or more plant-based texturizers or stabilizers; 80-98% by mass of the solvent; and 0.001-0.005% by mass of the one or more microorganism cultures. In another aspect, the composition comprises: 0.1-3.0% by mass of a flour from millet, sorghum, quinoa, amaranth, einkorn, teff, freekeh, farro (emmer), fonio, spelt, chia seeds, hemp hearts, buckwheat, or combinations thereof; 0.2-10.0% by mass of sunflower seed butter, coconut cream, pumpkin seed butter, watermelon seed butter, hemp seed butter, chia seed butter, flax seed butter, poppy seed butter, or combinations thereof; 0.1-0.75% by mass of one or more of locust bean gum, gellan gum, xanthan gum, acacia gum, tragacanth gum, guar gum, pectin, agar, or combinations thereof; 80-98% by mass of water; and 0.001-0.005% by mass of one or more of Bifidobacterium lactis, Lactobacillus acidophilus, Lactobacillus delbrueckii subsp. Bulgaricus, Lactobacillus delbrueckii subsp. Lactis, Lactobacillus helveticus, Lactobacillus kefiranofaciens, Lactobacillus paracasei, Lactobacillus plantarum, Lactococcus lactis, Leuconostoc spp., Pediococcus spp., Streptococcus thermophilus, Kazachstania unispora, Kluyveromyces lactis, Kluyveromyces marxianus, Saccharomyces cerevisiae, Saccharomyces fragilis, Torulaspora delbrueckii, or combinations thereof. In another aspect, the composition comprises: 0.1-3.0% by mass of a flour from millet, sorghum, or combinations thereof; 0.2-2.0% by mass of sunflower seed butter; 2-8% by mass of coconut cream; 0.1-0.75% by mass of one or more of locust bean gum, gellan gum, xanthan gum, or combinations thereof; 80-98% by mass of water; and 0001-0.005% by mass of one or more of Bifidobacterium lactis, Lactobacillus acidophilus, Lactobacillus delbrueckii subsp. Bulgaricus, Lactobacillus delbrueckii subsp. Lactis, Lactobacillus plantarum, or Streptococcus thermophilus, or combinations thereof. In another aspect, the composition is vegan, hypoallergenic and contains no added monosaccharides or disaccharides.

Another embodiment described herein is a method for manufacturing a composition described herein, the method comprising: combining and blending the one or more plant-based carbohydrate sources; the one or more plant seed butters or creams; the one or more plant-based texturizers or stabilizers; and the solvent; pasteurizing the blended composition; homogenizing the blended composition; cooling the homogenized composition; adding the one or more microorganism cultures to the blended, pasteurized, homogenized, and cooled composition and fermenting the composition until the pH is about 4.4 or lower; and cooling the fermented composition to about 4° C. In one aspect, the combined composition is blended at high shear until the composition is smooth and uniform. In another aspect, the blended composition is pre-heated prior to pasteurization. In another aspect, the blended composition is pasteurized using high temperature, short time (HTST) pasteurization or ultra-high temperature (UHT) pasteurization. In another aspect, the composition is homogenized at a high temperature. In another aspect, the composition is cooled to about ˜40° C. prior to adding the one or more microorganism cultures. In another aspect, the composition is fermented for about 4-8 hours at about ˜40° C. In another aspect, the composition is fermented until the pH is about 4.4 or lower.

Another embodiment described herein is a composition manufactured by any of the methods described herein.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A composition comprising:
   one or more plant-based carbohydrate sources;
   one or more plant seed butters or creams;
   one or more plant-based texturizers or stabilizers;
   a solvent; and
   one or more microorganism cultures.
Clause 2. The composition of clause 1, wherein the one or more plant-based carbohydrate sources comprises millet, sorghum, quinoa, amaranth, einkorn, teff, freekeh, farro (emmer), fonio, spelt, chia seeds, hemp hearts, buckwheat, or combinations thereof.
Clause 3. The composition of clause 1 or 2, wherein the one or more plant seed butters or creams comprises sunflower seed butter, coconut cream, pumpkin seed butter, watermelon seed butter, hemp seed butter, chia seed butter, flax seed butter, poppy seed butter, or combinations thereof.
Clause 4. The composition of any one of clauses 1-3, wherein the one or more plant-based texturizers or stabilizers comprises locust bean gum, gellan gum, xanthan gum, acacia gum, tragacanth gum, guar gum, pectin, agar, or combinations thereof.
Clause 5. The composition of any one of clauses 1-4, wherein the solvent comprises water.
Clause 6. The composition of any one of clauses 1-5, wherein the one or more microorganism cultures comprises one or more of Bifidobacterium lactis, Lactobacillus acidophilus, Lactobacillus delbrueckii subsp. Bulgaricus, Lactobacillus delbrueckii subsp. Lactis, Lactobacillus helveticus, Lactobacillus kefiranofaciens, Lactobacillus paracasei, Lactobacillus plantarum, Lactococcus lactis, Leuconostoc spp., Pediococcus spp., Streptococcus thermophilus, Kazachstania unispora, Kluyveromyces lactis, Kluyveromyces marxianus, Saccharomyces cerevisiae, Saccharomyces fragilis, Torulaspora delbrueckii, or combinations thereof.
Clause 7. The composition of any one of clauses 1-6, wherein the composition further comprises:
   one or more plant-based proteins comprising pea protein concentrate, potato protein concentrate, bean protein concentrate, sunflower seed protein concentrate, rapeseed protein or combinations thereof; and/or
   one or more fruit and vegetable soluble fiber concentrates or pomaces or combinations thereof.
Clause 8. The composition of any one of clauses 1-7, wherein the composition comprises:
   0.1-3.0% by mass of the one or more plant-based carbohydrate sources;
   0.2-10% by mass of the one or more plant seed butters or creams;
   0.1-0.75% by mass of the one or more plant-based texturizers or stabilizers;
   80-98% by mass of the solvent; and
   0.001-0.005% by mass of the one or more microorganism cultures.
Clause 9. The composition of any one of clauses 1-9, wherein the composition comprises:
   0.1-3.0% by mass of a flour from millet, sorghum, quinoa, amaranth, einkorn, teff, freekeh, farro (emmer), fonio, spelt, chia seeds, hemp hearts, buckwheat, or combinations thereof;
   0.2-10% by mass of sunflower seed butter, coconut cream, pumpkin seed butter, watermelon seed butter, hemp seed butter, chia seed butter, flax seed butter, poppy seed butter, or combinations thereof;
   0.1-0.75% by mass of one or more of locust bean gum, gellan gum, xanthan gum, acacia gum, tragacanth gum, guar gum, pectin, agar, or combinations thereof;
   80-98% by mass of water; and
   0.001-0.005% by mass of one or more of Bifidobacterium lactis, Lactobacillus acidophilus, Lactobacillus delbrueckii subsp. Bulgaricus, Lactobacillus delbrueckii subsp. Lactis, Lactobacillus helveticus, Lactobacillus kefiranofaciens, Lactobacillus paracasei, Lactobacillus plantarum, Lactococcus lactis, Leuconostoc spp., Pediococcus spp., Streptococcus thermophilus, Kazachstania unispora, Kluyveromyces lactis, Kluyveromyces marxianus, Saccharomyces cerevisiae, Saccharomyces fragilis, Torulaspora delbrueckii, or combinations thereof.
Clause 10. The composition of any one of clauses 1-9, wherein the composition comprises:
   0.1-3.0% by mass of a flour from millet, sorghum, or combinations thereof;
   0.2-2.0% by mass of sunflower seed butter;
   2-8% by mass of coconut cream;
   0.1-0.75% by mass of one or more of locust bean gum, gellan gum, xanthan gum, or combinations thereof;
   80-98% by mass of water; and
   0.001-0.005% by mass of one or more of Bifidobacterium lactis, Lactobacillus acidophilus, Lactobacillus delbrueckii subsp. Bulgaricus, Lactobacillus delbrueckii subsp. Lactis, Lactobacillus plantarum, or Streptococcus thermophilus, or combinations thereof.
Clause 11. The composition of any one of clauses 1-11, wherein the composition is vegan, hypoallergenic and contains no added monosaccharides or disaccharides.
Clause 12. A method for manufacturing the composition of any one of clauses 1-11, the method comprising:
   combining and blending the one or more plant-based carbohydrate sources; the one or more plant seed butters or creams; the one or more plant-based texturizers or stabilizers; and the solvent;
   pasteurizing the blended composition;
   homogenizing the blended composition;
   cooling the composition;
   adding the one or more microorganism cultures to the blended, pasteurized, homogenized, and cooled composition and fermenting the composition until the pH is about 4.4 or lower; and
   cooling the fermented composition to about 4° C.
Clause 13. The method of clause 12, wherein the combined composition is blended at high shear until the composition is smooth and uniform.
Clause 14. The method of clause 12 or 13, wherein the blended composition is pre-heated prior to pasteurization.
Clause 15. The method of any one of clauses 12-14, wherein the blended composition is pasteurized using high temperature, short time (HTST) pasteurization or ultra-high temperature (UHT) pasteurization.
Clause 16. The method of any one of clauses 12-15, wherein the composition is homogenized at a high temperature.
Clause 17. The method of any one of clauses 12-17, wherein the homogenized composition is cooled to about 40° C. prior to adding the one or more microorganism cultures.
Clause 18. The method of any one of clauses 12-16, wherein the composition is fermented for about 4-8 hours at about ˜40° C.
Clause 19. The method of any one of clauses 12-17, wherein the composition is fermented until the pH is about 4.4 or lower.
Clause 20. A composition manufactured by the method of any one of clauses 12-18.

### EXAMPLES

### Example 1

A plant-based dairy alternative composition was prepared comprising the following components shown in Table 2. In one aspect, the plant-based dairy alternative can be used as a buttermilk alternative. Alternative uses are for the preparation of yogurt or kefir.

TABLE 2
Exemplary Plant-Based Dairy Alternative Composition

| Component | Mass Percentage (%) |
|---|---|
| Millet and sorghum flours | 0.2-0.8 |
| Sunflower seed butter | 1-1.5 |
| Coconut cream | 3-5 |
| Locust bean gum, gellan gum, xanthan gum | 0.2-0.5 |
| Water | 92-95 |
| Microorganism cultures | 0.002-0.004 |

The microorganism cultures comprised at least one or more of Bifidobacterium lactis, Lactobacillus acidophilus, Lactobacillus delbrueckii subsp. Bulgaricus, Lactobacillus delbrueckii subsp. Lactis, Lactobacillus plantarum, or Streptococcus thermophilus.

The composition was manufactured by combining all components except the microorganism cultures into a high-shear mixer, blending the batch at high shear until it is smooth and the gums were dispersed, and no clumps were formed. The composition was transferred to a batch kettle and mixed with gentle agitation until the pasteurization/homogenization steps. Prior to pasteurization, the composition was preheated to 40-48° C. (107-117° F.). Pasteurization was performed at 93° C. (200° F.) followed by homogenization at 93° C. (200° F.), 500/1500 psi for 7 minutes.

Following pasteurization/homogenization, the mixture was cooled to about 40° C. (106° F.). The microorganism cultures were added and mixed and the composition was allowed to ferment at ˜40° C. (106° F.) for about 5-8 hours or until the pH reached 4.4 or lower. See FIG. 23. Following culturing, the composition was transferred to a cooling tank and held at 4° C. (39° F.).

Developers of commercial cultured products made from plant-based ingredients have struggled to reduce culturing times to those similar to cultured dairy products which are typically 6 to 8 hours. They have had to resort to adding monosaccharides and disaccharides to reduce these culturing times. Even with the addition of monosaccharides and disaccharides, these culturing times still typically exceed those of cultured dairy products. The advantage of the compositions and method described herein is the addition of a small amount, 0.1-3.0%, of ancient grain flours, without adding supplemental monosaccharides or disaccharides, that enhances the culturing times of the plant-based dairy alternatives described herein to be similar to those of cultured dairy products. See FIG. 23.

### Example 2

A plant-based yogurt composition was prepared comprising the following components shown in Table 3. The plant-based dairy alternative from Example 1 was combined with a fruit preparation to create yogurts. Exemplary fruit preparations include strawberry, blueberry, raspberry, mixed berry, peach, mango, pineapple, etc.

TABLE 3
Exemplary Plant-Based Yogurt Composition

| Component | Mass Percentage (%) |
|---|---|
| Plant-Based Dairy Alternative (from Example 1) | 80-90% |
| Fruit preparation (e.g., strawberry, etc.) | 10-20% |

The yogurt was prepared by combining the plant-based dairy alternative with the fruit preparation at the specified mass percentages. One product is a drinkable version of yogurt. Another product is a solid yogurt that comprises additional thickeners added to the plant-based dairy alternative and fruit preparation mixture to increase the solidity. The products are dispensed into receptacles for storage and marketing.

What is claimed:

1. A composition comprising:
one or more plant-based carbohydrate sources;
one or more plant seed butters or creams;
one or more plant-based texturizers or stabilizers;
a solvent; and
one or more microorganism cultures.

2. The composition of claim 1, wherein the one or more plant-based carbohydrate sources comprises millet, sorghum, quinoa, amaranth, einkorn, teff, freekeh, farro (emmer), fonio, spelt, chia seeds, hemp hearts, buckwheat, or combinations thereof.

3. The composition of claim 1, wherein the one or more plant seed butters comprises sunflower seed butter, coconut cream, pumpkin seed butter, watermelon seed butter, hemp seed butter, chia seed butter, flax seed butter, poppy seed butter, or combinations thereof.

4. The composition of claim 1, wherein the one or more plant-based texturizers or stabilizers comprises locust bean gum, gellan gum, xanthan gum, acacia gum, tragacanth gum, guar gum, pectin, agar, or combinations thereof.

5. The composition of claim 1, wherein the solvent comprises water.

6. The composition of claim 1, wherein the one or more microorganism cultures comprises one or more of Bifidobacterium lactis, Lactobacillus acidophilus, Lactobacillus delbrueckii subsp. Bulgaricus, Lactobacillus delbrueckii subsp. Lactis, Lactobacillus helveticus, Lactobacillus kefiranofaciens, Lactobacillus paracasei, Lactobacillus plantarum, Lactococcus lactis, Leuconostoc spp., Pediococcus spp., Streptococcus thermophilus, Kazachstania unispora, Kluyveromyces lactis, Kluyveromyces marxianus, Saccharomyces cerevisiae, Saccharomyces fragilis, Torulaspora delbrueckii, or combinations thereof.

7. The composition of claim 1, wherein the composition further comprises:
one or more plant-based proteins comprising pea protein concentrate, potato protein concentrate, bean protein concentrate, sunflower seed protein concentrate, rapeseed protein or combinations thereof; and/or
one or more fruit and vegetable soluble fiber concentrates or pomaces or combinations thereof.

8. The composition of claim 1, wherein the composition comprises:
0.1-3.0% by mass of the one or more plant-based carbohydrate sources;
0.2-10% by mass of the one or more plant seed butters or creams;
0.1-0.75% by mass of the one or more plant-based texturizers or stabilizers;
80-98% by mass of the solvent; and
0.001-0.005% by mass of the one or more microorganism cultures.

9. The composition of claim 1, wherein the composition comprises:
0.1-3.0% by mass of a flour from millet, sorghum, quinoa, amaranth, einkorn, teff, freekeh, farro (emmer), fonio, spelt, chia seeds, hemp hearts, buckwheat, or combinations thereof;
0.2-10% by mass of sunflower seed butter, coconut cream, pumpkin seed butter, watermelon seed butter, hemp seed butter, chia seed butter, flax seed butter, poppy seed butter, or combinations thereof;
0.1-0.75% by mass of one or more of locust bean gum, gellan gum, xanthan gum, acacia gum, tragacanth gum, guar gum, pectin, agar, or combinations thereof;
80-98% by mass of water; and
0.001-0.005% by mass of one or more of Bifidobacterium lactis, Lactobacillus acidophilus, Lactobacillus delbrueckii subsp. Bulgaricus, Lactobacillus delbrueckii subsp. Lactis, Lactobacillus helveticus, Lactobacillus kefiranofaciens, Lactobacillus paracasei, Lactobacillus plantarum, Lactococcus lactis, Leuconostoc spp., Pediococcus spp., Streptococcus thermophilus, Kazachstania unispora, Kluyveromyces lactis, Kluyveromyces marxianus, Saccharomyces cerevisiae, Saccharomyces fragilis, Torulaspora delbrueckii, or combinations thereof.

10. The composition of claim 1, wherein the composition comprises:
0.1-3.0% by mass of a flour from millet, sorghum, or combinations thereof;
0.2-2.0% by mass of sunflower seed butter;
2-8% by mass of coconut cream;
0.1-0.75% by mass of one or more of locust bean gum, gellan gum, xanthan gum, or combinations thereof;
80-98% by mass of water; and
0.001-0.005% by mass of one or more of Bifidobacterium lactis, Lactobacillus acidophilus, Lactobacillus delbrueckii subsp. Bulgaricus, Lactobacillus delbrueckii subsp. Lactis, Lactobacillus plantarum, or Streptococcus thermophilus, or combinations thereof.

11. The composition of claim 1, wherein the composition is vegan, hypoallergenic and contains no added monosaccharides or disaccharides.

12. A method for manufacturing the composition of claim 1, the method comprising:
combining and blending the one or more plant-based carbohydrate sources; the one or more plant seed butters or creams; the one or more plant-based texturizers or stabilizers; and the solvent;
pasteurizing the blended composition;
homogenizing the blended composition;
cooling the homogenized composition;
adding the one or more microorganism cultures to the blended, pasteurized, homogenized, and coiled composition and fermenting the composition until the pH is about 4.4 or lower; and
cooling the fermented composition to about 4° C.

13. The method of claim 12, wherein the combined composition is blended at high shear until the composition is smooth and uniform.

14. The method of claim 12, wherein the blended composition is pre-heated prior to pasteurization.

15. The method of claim 12, wherein the blended composition is pasteurized using high temperature, short time (HTST) pasteurization or ultra-high temperature (UHT) pasteurization.

16. The method of claim 12, wherein the composition is homogenized at a high temperature.

17. The method of claim 12, wherein the homogenized composition is cooled to about ˜40° C.

18. The method of claim 12, wherein the composition is fermented for about 4-8 hours at about ˜40° C.

19. The method of claim 12, wherein the composition is fermented until the pH is about 4.4 or lower.

20. A composition manufactured by the method of claim 12.
